(19) United States
(12) Patent Application Publication
(10) Pub. No.: US 2009/0318713 A1
(43) Pub. Date: Dec. 24, 2009

(54) PROCESS FOR MANUFACTURING A CYCLIC DIESTER OF AN A-HYDROXY ACID

(75) Inventor: Samuel J. WAJC, Haifa (IL)

Correspondence Address: OBLON, SPIVAK, MCCLELLAND MAIER & NEUSTADT, L.L.P., 1940 DUKE STREET, ALEXANDRIA, VA, 22314, US

(73) Assignee: KTANTON Ltd., Haifa (IL)

(21) Appl. No.: 12/483,309

(22) Filed: Jun. 12, 2009

(30) Foreign Application Priority Data
Jun. 12, 2008 (EP) 08158153.0

(51) Int. Cl. C07D 309/32

(52) U.S. Cl. 549/231

(57) ABSTRACT

Process for manufacturing a cyclic diester of an α-hydroxy acid comprising the following steps:
a substantially anhydrous salt of a divalent metal and of an α-hydroxy acid is mixed with a strong acid, the pKa of which is less than that of the α-hydroxy acid and the salt of which with the divalent metal is hygroscopic; and
the mixture is left to react for a sufficient time in order to obtain the cyclic diester dispersed in the hygroscopic salt.

### BACKGROUND OF THE INVENTION

The present application relates to a process for manufacturing a cyclic diester of an α-hydroxy acid and in particular, lactide or glycolide. It relates, in particular, to the manufacture of lactide (LD2 or Lactide Diester) which is a key intermediate for the manufacture of lactic acid polymers (PLA or Polylactide), a bio-sourced (i.e. based on natural resources) and biodegradable polymer.

The production of PLA is currently developing rapidly. However, the conventional processes for producing LD2 use lactic acid (L1A) as a reactant and amount to a series of difficult condensation, water separation and depolymerization steps. These steps are energy intensive, and the lactide formed is not pure. Furthermore, the degree of conversion is not above 60/70% currently with, in addition, by-products, the end-of-life of which must subsequently be managed. The synthesis of the lactide is the most important step in the conventional PLA manufacturing process. It is this step that will govern the price of the final polymer. The lactide must be as pure as possible in order to be able then to carry out the ring-opening polymerization.

The principle on which the present invention is based consists in using a lactate (or another α-hydroxy acid salt) that is reacted with a strong acid in order to release lactic acid (or another α-hydroxy acid) and to form a dehydrating (hygroscopic) salt. The latter will capture in situ the two molecules of water that will be released during the various reactions that lead to the formation of the diester (and which will be explained in detail further on). The dehydrating salt could be subsequently reused as fertilizer or for therapeutic applications. Thus a process with useful and environmentally by-products is obtained which consumes less energy than the processes of the prior art, the use of a dehydrating salt limiting the temperature required in order to eliminate the water of reaction and preventing, in addition, the oligomerization of the α-hydroxy acid.

In order for this principle to work, the Applicant observed that it was necessary for the pKa of the strong acid to be lower than that of the α-hydroxy acid (so that this strong acid can effectively react with the organic salt and release the α-hydroxy acid) and for the resulting salt to be dehydrating.

### BRIEF SUMMARY OF THE INVENTION

The present invention relates to processes for manufacturing a cyclic diester of an α-hydroxy acid comprising:
a salt of a metal and of an α-hydroxy acid is mixed with a strong acid, the pKa of which is less than that of the α-hydroxy acid and the salt of which with the metal is hygroscopic; and
the mixture is left to react at a sufficient temperature and for a sufficient time in order to obtain the cyclic diester dispersed in the hygroscopic salt.

### BRIEF DESCRIPTION OF SEVERAL VIEWS OF THE DRAWING

FIG. 1 shows NMR analyses of crystals of Example 1.

FIG. 2 shows the change in setpoint temperature and the change in the product of the accumulated sublimate mass (m. cum., in g) times 10, over time for the product of Example 2.

FIG. 3 shows the accumulated weight of sublimate (x-axis) and the weight fraction of L1A, LD and L2A (y-axis) for the product of Example 2.

FIG. 4 shows the change in setpoint temperature and the change in the product of the accumulated sublimate mass (m. cum., in g) times 10, over time for the product of Example 3.

FIG. 5 shows the average concentrations (x or mass fraction) for L1A, LD2, and L2A.

FIG. 6 shows that, under a pressure of 5 to 10 mbar, the separation can be carried out at a relatively low temperature.

FIG. 7 shows the concentrations obtained by analysis of the NMR signals by the two routes described in the previous example.

FIG. 8 shows the results for Example 4 repeated at a higher temperature.

FIG. 9 shows that the lactide content of the sublimate was increased much more rapidly for Example 5 than in the previous example.

### DETAILED DESCRIPTION OF THE INVENTION

The present invention therefore relates to a process for manufacturing a cyclic diester of an α-hydroxy acid comprising the following steps:
a salt of a metal and of an α-hydroxy acid is mixed with a strong acid, the pKa of which is less than that of the α-hydroxy acid and the salt of which with the metal is hygroscopic; and
the mixture is left to react at a sufficient temperature and for a sufficient time in order to obtain the cyclic diester dispersed in the hygroscopic salt.

The α-hydroxy acid (AHA) in question in the context of the invention is preferably obtained by fermentation of natural products such as the sugars from plants, fruit, etc. or dairy products. It is preferably lactic, glycolic, glutaric, mandelic, malic, citric or tartaric acid, the first two being preferred. Lactic acid gives good results.

The metal in question in the context of the invention must be such that the corresponding salt of the strong acid is dehydrating; it may be chosen from Fe, Mg, Zn, Al and Ca. It is preferably a divalent metal. Alkaline-earth type metals are preferred. Ca and Mg are particularly preferred since they can give, inter alia, recyclable by-products.

The metal α-hydroxy acid salts used in the process according to the invention are not generally available commercially as such salts. On the other hand, hydrates based on Mg and Ca lactate are available respectively under the trade names PURAMEX® MG (dihydrate of MgLa2 or Mg lactate) and PURACAL® (pentahydrate of CaLa2 or Ca lactate). Alternatively, such hydrates may be synthesized by fermentation of sugars or dairy products using bacteria (with addition of a base in order to obtain a pH close to (or even slightly below) 7), then purification by crystallization. In the case of glycolates, glycolic acid is on the market, which can be reacted with a metal hydroxide in order to form a hydrate of the corresponding glycolate.

The aforementioned hydrates are easily (at least partially) dehydrated (to form a powder of anhydrous crystals) at a moderate temperature, even at atmospheric pressure. For example, treatment at a temperature between 90 and 180° C., preferably between 100 and 160° C. and most particularly preferably between 110 and 130° C. gives good results (generally even at the end of one to two hours treatment at atmospheric pressure). Too low a temperature needlessly increases the treatment time and it has been observed that too high a temperature limits the porosity of the crystals obtained and therefore, consequently, does not favour their reaction with the strong acid. The degree of dehydration can be checked by weighing. The dehydrated salt is then preferably cooled and kept in a desiccator before use.

It should be noted that the aforementioned dehydration step is optional. Specifically, it has been observed that it suffices for the medium to contain sufficient dehydrating agent and/or for the mixture to be left “to dry” for a long enough time in order to eliminate all the water. It is however preferable to start from an anhydrous salt.

Most particularly preferably, the metal salt is an anhydrous divalent metal salt. In this case, during the reaction with the strong acid, 2 molecules of the AHA will be brought together and the dehydrating agent (salt) formed will promote the formation of the cyclic diester by capturing the 2 molecules of water formed.

The strong acid used in the process according to the invention (and which is also preferably anhydrous) must fulfil two conditions:
its pKa must be below that of the α-hydroxy acid; and
its salt with the divalent metal must be hygroscopic, i.e. be capable of reacting with water to shift the equilibrium towards the formation of the lactide; generally, this takes place via the in situ formation of hydrates. In order to be able to separate the cyclic diester therefrom by distillation (sublimation: see further on), the hydrate formed in situ will preferably have a decomposition temperature above the distillation temperature of the cyclic diester.

Acids that are particularly suitable are sulphuric or phosphoric acid or anhydride (considered to be an acid in the context of the invention). The 1^{st} has the advantage that its pKa2 is also low (1.9), so that it is generally also below the pKa of the α-hydroxy acid (which is, for example 3.86 for lactic acid and 3.83 for glycolic acid) and that a single mole of acid per mole of metal salt suffices. It has however the drawbacks of resulting in an LD2 that is sometimes coloured and of resulting in by-products that are difficult to recycle (CaSO₄, MgSO₄). Phosphoric acid (pKa1=2.1) on the other hand does not result in a coloration problem and makes it possible to obtain by-products that are more easily recyclable (in particular when based on Ca or Mg). On the other hand, since its pKa2 is relatively high (7.2), it is generally above the pKa of the α-hydroxy acid. Hence, it is preferable to mix 2 moles of this acid (or one mole of the corresponding anhydride, i.e. P₂O₅) per mole of divalent metal salt.

The mixing of the metal salt with the strong acid may be carried out in any known manner that makes it possible to achieve intimate mixing of the reactants, for example in a mortar, a mixer, a mixing and grinding machine or an extruder. The latter method gives good results in practice since it makes it possible to directly obtain a product (rod, or granules if the rod is cut on exiting the extruder) having a high surface area/volume ratio enabling the heat generally used to promote the dehydration to penetrate inside it. Typically, the extrusion of a rod having a diameter of 2 to 5 mm gives good results. Alternatively, the mixture from another type of mixer may be shaped into a crepe (film) or into particles before being subjected to a maturing operation (complete reaction).

Preferably, the acid is added slowly to the metal salt to avoid having too violent a heating which could lead to problems of degradation of the reaction products. For this same reason, the acid is preferably not added to the reaction medium in excess.

Depending on the choice of reactants, an additional desiccant (hygroscopic salt) (which desiccant may or may not be of the same nature as the salt) may be added to the reaction medium either during or after the mixing of the reactants. Indeed, when the mixture is prepared in an extruder, its consistency must optionally be adapted (in order to obtain an effectively extrudable paste). Moreover, depending on the hygroscopic capacity of the salt (and especially: on the number of water molecules that the hydrates which it is capable of forming comprise), an external supply of salt may be required in order to be able to react with (absorb) all the water of reaction. Hence, in one particularly advantageous variant, a salt from a previous production run (batch) may be partly re-used.

Similarly, it may be advantageous to add to the mixture (preferably before its reaction), another by-product from a previous batch, for example AHA which might not have been converted to diester. Proceeding in such a way makes it possible to increase the overall yield of the reaction (the acid produced in one batch being recycled to the following batch).

As mentioned above, the mixture (which is generally in the form of a paste) must be subjected to a maturing operation, i.e. it is necessary to leave it to rest for the time needed so that the succession of chemical reactions leading to the cyclic diester (release of the α-hydroxy acid, followed by its dimerization (to lactoyl lactate or L2A) and then by its cyclization (to LD2), each time with release of water) can be carried out to term. It is especially necessary to ensure that the water of reaction is evacuated from the mixture in order to avoid polycondensing the α-hydroxy acid. It is also necessary to ensure that the temperature does not rise too much, in order to avoid distilling said acid, and by doing so extracting it prematurely from the reaction medium. Conditions identical to those for the drying of the hydrates explained above are particularly suitable. Preferably, the paste resulting from the mixture (in the form of rod, granules, film, etc.) is deposited onto a bed of pulverulent desiccant during this step so that this desiccant can capture the water and to check, on the one hand, that the reactions are definitely shifted to the right and, on the other hand that the AHA does not oligomerize. This powder also has a mechanical role, namely preventing caking during the following step of separating the diester.

In the particular case where the AHA is lactic acid, if water is not correctly removed from the reaction medium, this can become the site of a certain hydrolysis of the LD2, either to lactoyl lactic acid (L2A), or to the monomer L1A. It is possible to limit this effect by adding to the mixture before extrusion an amount to be determined of desiccant, for example of the secondary product of a previous batch that will have been dried at 250° C. for example. It is also possible to add a commercial desiccant, such as anhydrous Mg sulphate.

At the end of the maturing step, a hard but friable solid is generally present, which it is advantageous to coarsely mill (optionally with prior cooling) in order to facilitate the separation of the diester. Indeed, since the water of reaction has been gradually (absorbed or) captured by the hygroscopic salt, this water has caused the agglomeration of the crystals in the course of forming a compact mass. This is in particular the case when the desiccant is Mg hydrogen phosphate (i.e. when starting from MgLa and a source of phosphate ions), since this is a well-known magnesium cement. The milling of this mass may be carried out in any known manner, preferably until coarse grains having a size of around 3 to 5 mm in diameter are obtained. On one hand, this limits the resistance to material transfer in the case where the diester is separated by distillation apart from the residual porous mass. On the other hand, this size is characteristic of fertilizer granules, so that this by-product could optionally be used as fertilizer without having to modify the spreading machines.

The grains thus obtained are then generally subjected to a separation (extraction) of the diester. This extraction may take place by distillation (sublimation) under vacuum or a stream of inert gas, by liquid-solid extraction, etc. Vacuum distillation in a suitable column, preferably a rotating column (to create a stirring effect) that is heated (for example at a final temperature (see above) from 120 to 240° C., preferably from 150 to 200° C.), gives good results. In the case where the AHA is added to the mixture, it may be advantageous to work at a higher temperature, for example at 180° C. and above, or even up to a few degrees below the racemization temperature of the diester, which it is better not to exceed (and which is 250° C. in the case of LD). In this variant, it may be advantageous to fluidize the particles (promote the relative movement thereof) by addition of desiccant particles.

To promote the elimination of the residual water, it is preferable to very slowly increase the temperature during the first part of the distillation (up to 125° C.).). Thus, the temperature will be allowed, for example, to increase at a rate of 1 to 2° C./min, without decreasing the pressure below 50 to 100 mbar. Premature evaporation of the AHA (which would not therefore have had the chance to be converted to the diester) will thus be avoided. As the volatility of the water is much higher than that of the organic products (diester, AHA (which has not completely reacted) and oligomers), the acid reaction medium will be increasingly favourable to the displacement of the equilibrium in favour of the diester. Since, at these temperatures, these organic products are generally molten, the mobility necessary for the reaction is generally acquired.

Also for this distillation step, it is advantageous to start from hard, porous granules, the pores of which are initially plugged by the product that it is desired to distil (diester). If the pores are quite narrow, the liquid will be held therein by capillary action. As the heating is necessarily carried out from the outside towards the inside of each granule, its surface has a tendency to dry out during the distillation. Hence, for this distillation use could be made of industrial equipment (sublimers, for example) filled with a mixture of partially dried granules and fresh granules, where the risk of caking would be nonexistent. At the same time, the vapours from the distillation (or sublimation) would be condensed in a heat exchanger of appropriate shape.

At the end of this step, the following will therefore have been recovered: on the one hand, a sublimate, the various constituents of which (mainly the diester and, as impurities, the residual monomer (AHA) and the dimer essentially) will have to be separated, preferably by fractional melting or fractional distillation (resublimation); and, on the other hand, a solid residue that is mainly composed of the dehydrating salt and of higher oligomers of AHA.

The sublimation operation may take place in a heat exchanger comprising a chamber and a bundle of tubes through which a heat transfer fluid flows, which heat transfer fluid is first brought to a temperature slightly (typically 2 to 10° C.) below the crystallization temperature of the ester that it is desired to recover, and where the vapours from the sublimation will be condensed. An annular crust of crude ester containing, in particular, impurities that are more volatile than the ester itself, such as the corresponding AHA, will thus be deposited on each tube of said cooled bundle during the heating of the grains of solid mentioned above.

It then suffices to slowly increase the temperature of the heat transfer fluid in order to give rise to the preferential melting of a mixture rich in impurities (since its melting point is lower than that of the pure diester). This viscous mixture flows slowly by gravity and is therefore recovered separately in the lower part of the exchanger. It then suffices to increase the temperature of the heat transfer fluid sufficiently in order to melt all of the remaining crystalline layer, i.e. purified diester, which will then also be recovered in the lower part of the exchanger and optionally subjected to a second, or even to a 3^{rd} similar fractional crystallization treatment, preferably in another apparatus.

Particularly advantageously, the sublimation treatment takes place in a heat exchanger comprising a chamber and two bundles of tubes, which are preferably vertical and overlapping or in staggered rows, and its chamber is designed to be able to operate under vacuum. Thus, instead of heating the layer of crude ester deposited on the tubes of one of the bundles beginning with its coldest zone (by heating the coolant), which has the effect of weakening the adhesion of said layer to the bundle of tubes, it is possible to send, into the second tube bundle, a hot heat transfer fluid which will heat, essentially by radiation, the part of the crude ester crust that is still hot. The viscous liquid resulting from the melting of the last layer of crude ester exposed will drip onto a solid ester support whilst the vacuum will be maintained so as to simultaneously allow the fractional distillation of the volatile impurities contained in the crude diester. The purified liquid diester will be recovered in the lower part of the exchanger, and the volatile portions will be condensed elsewhere.

The same principle may be applied in order to block the intake of non-volatile impurities into the crude diester layer. For this purpose, it is possible to place a small heat exchanger between the sublimer containing the grains of solid to be treated and the bundle or bundles of tubes, this small heat exchanger being raised to a temperature above that of the desublimation of the diester but below that of the condensation of the oligomers of this diester, so as to recover a condensate of oligomers to be hydrolysed before the recycling thereof.

A single laboratory device that makes it possible to carry out all these operations in succession is a BÜCHI device of glass oven type. Such a device comprises a rotating oven that is preferably transparent (for example made of glass) that has a generally tubular shape comprising ball tubes connected by parts of smaller diameter, and which is extended and/or connected to a container that is preferably also transparent and cooled (condenser). A rotary seal at the cold end of the condenser makes it possible to connect the device to a vacuum pump, the water released during the reaction that is not captured by the dehydrating salt being condensed in a dry-ice trap that protects said vacuum pump.

For its use in the process that is the subject of the invention, this device may be used as follows: the sublimer (or evaporator) is loaded with a few tens of grams of granules, and optionally with a small amount of pulverulent desiccant. The ball tubes intended to serve as a condenser are connected to the evaporator. The assembly is slid into place, the evaporator alone being in the heating chamber. The condenser is connected to the vacuum pump and the assembly is put under a partial vacuum. The evaporator and condenser are rotated so that the granules roll over one another, periodically coming into contact with the (heated) wall of the evaporator. The pressure can be set at 5 to 10 mbar and the temperature can be increased slowly up to 125° C. (1 to 2° C./min). During this time, water is evacuated and recovered in the dry-ice trap that protects the vacuum pump.

It is then possible to increase the setpoint temperature and to give rise to the distillation of the organic products occluded in the granules. Almost all of the vapours released are deposited in the condenser cooled by the ambient air. A small portion escapes to the dry-ice trap, whilst a portion of the compounds that are less volatile than the diester is condensed near the neck for passing from the evaporator to the condenser.

The operation of the device can be interrupted as required, separate the condenser, replace it with a clean condenser and then restart the device. Thus the entire amount of crude ester sublimed since the last restart is recovered, an amount that can be weighed and analysed.

The company LIST sells industrial versions of sublimers-desublimers, the operating principle of which is similar to that described above.

Alternatively, it is possible to use a vertical oven loaded with desiccant granules containing the diester and that preferably operate at atmospheric pressure. A hot inert gas feeds the device so that a wave of heat runs through the stack. This gives rise to the creation of a wave of material transfer so that:

a. —the organic mixture is in the granules before the passage of the wave;
b. —there is gradual evaporation during the passage of the wave;
c. —the granules are depleted after the passage of the wave.

The initial temperature of the granules is preferably close to 100° C. so as to avoid any condensation in the stack. The inlet temperature of the inert gas is preferably below that which would lead to the degradation of the diester, but high enough so that the partial pressure of the vapour of the diester is relatively high. Thus, in the case of the production of lactide, the inlet temperature of the inert gas may be between 160 and 210° C.

The inert gas, loaded with vapour, is directed to a heat exchanger having a single vertical tube bundle where the desublimation takes place. Since this desublimer operates at atmospheric pressure, the purification of the crude diester may be carried out slightly differently to that explained above.

Indeed, once the entire stack of granules is depleted, and when all the (impure) diester has been attached to the tubes of the heat exchanger in the form of sublimate, the intake of hot gas at the inlet of the stack is interrupted. A new stream of inert gas is then introduced into the base of the desublimer, gas brought to a temperature slightly (from 5 to 15° C. for example) above the melting point of the diester. In that way, the (hottest) outer layer of the sublimate is melted. The viscous liquid thus produced drips onto the surface of the deposition that is still solid (and cold) and simultaneously undergoes a desorption of the compounds that are more volatile than the diester. The latter are condensed elsewhere, the condensate being recycled to an operation for preparing the salt of the AHA. The purified diester is recovered at the base of the heat exchanger. Finally, the inert gas depleted of organic vapours is recycled to the heat exchanger where it is brought to the desired temperature.

The present invention is illustrated nonlimitingly by Examples 1 to 5 below.

### Example 1

In this example, the following reaction has in fact been carried out:

MgLa₂+2H₃PO₄.H₂O+Ca(H₂PO₄)₂→Mg(H₂PO₄)₂.2H₂O+LD+Ca(H₂PO₄)₂.2H₂O

In order to do this, the procedure below was followed:

Magnesium lactate dihydrate (bought from PURAC under the trade name PURAMEX® MG) was dried by static heating at atmospheric pressure at 130° C. for 7 hours. The dehydrated salt was then kept in a desiccator.

20.2 g of this anhydrous Mg lactate were mixed with 17 g of (Ca(H₂PO₄)₂ (previously dehydrated at 110° C. under atmospheric pressure over 12 h in an oven). 23.2 g of 85% technical grade phosphoric acid were then gradually poured over the powder, while mixing in a mortar. In this step the phosphoric acid released lactic acid. The paste obtained was white and very thick.

This paste was put in an oven for 12 h at 80° C. under atmospheric pressure. The hard solid obtained was milled into grains that were introduced into a distillation flask. The latter was heated in an oil bath at 180° C. and under 4 mbar for 5 h.

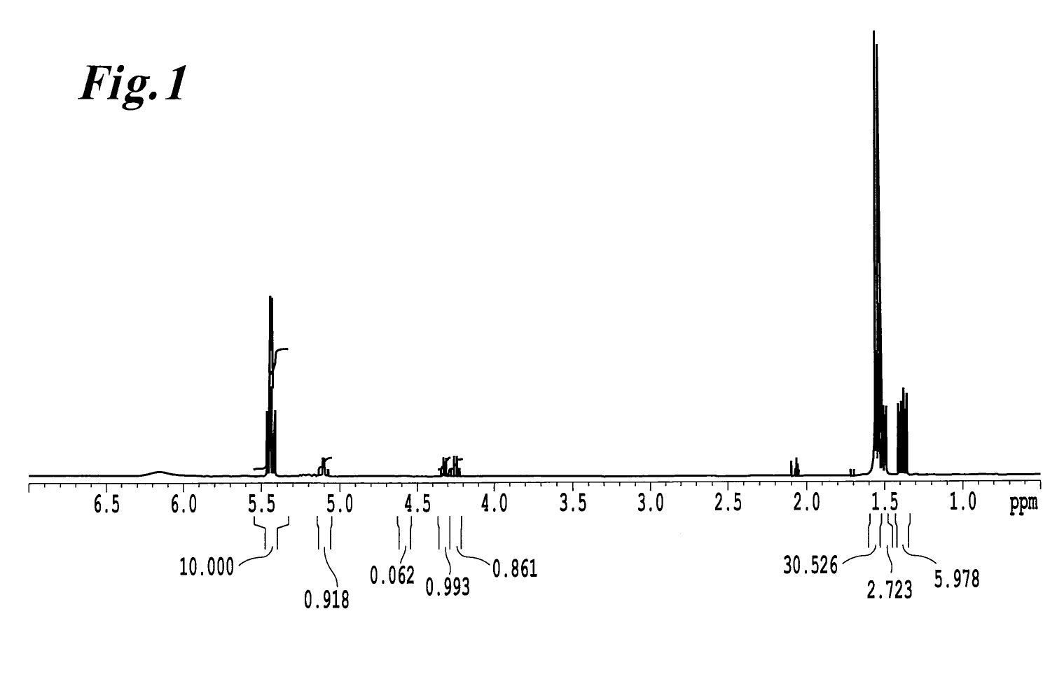

Crystals were obtained on the walls of the glassware. 2.7 g of these crystals were recovered. NMR (nuclear magnetic resonance) analyses were carried out on these crystals (see FIG. 1) and revealed that 2.7 g of 85% pure lactide had been extracted, the 15% impurity being 7.3% lactic acid and 8.4% open dimer. Therefore a yield by mass of the order of 20% was obtained.

Here is the protocol used for the NMR analyses:
20 mg of the sample were withdrawn;
it was put into solution in acetone;
the insolubles of the solution were dispersed using ultrasounds;
the solution was filtered in order to remove the insolubles therefrom before analysing it by the NMR technique.

### Example 2

The purpose of this example is to demonstrate that the distillation allows the acid to be effectively separated from the diester.

In order to do this, the procedure below was followed:

13.9 g of CaLa pentahydrate and 10.5 g of lactic acid (LA, 88%) were mixed in a Petri dish. The mixture was partially dehydrated under atmospheric pressure at 110° C. over 90 min. The solid obtained, which weighed 19.3 g, was finely milled and 18.8 g of powder was recovered, with which 12.2 g of phosphoric acid (85% aqueous solution) were mixed in a mortar.

The resulting paste was extruded using a syringe to form a sort of rod (spaghetti) weighing 26.9 g that was dried in a Petri dish for 3.5 h under atmospheric pressure at 110° C. The solid, dried and coarsely milled to particles of 2 to 5 mm then weighed 23.4 g.

2 g of a previous batch (produced under identical conditions) were added thereto and a BÜCHI type distillation device (as described previously) was loaded with the 25.4 g of solid thus obtained.

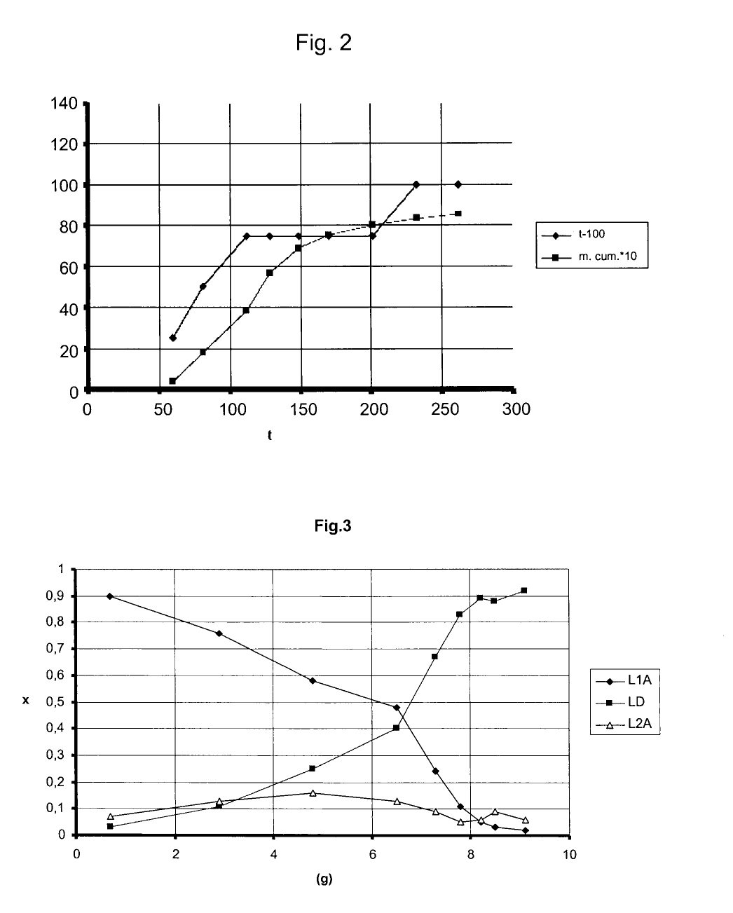

The device was put under vacuum (5 to 10 mbar) and the setpoint temperature was changed as indicated in FIG. 2 (which in fact represents, on the y-axis, this temperature −100° C.).

At regular intervals, the rotation of the device and the heating were stopped in order to be able to recover sublimate (strictly speaking, at the start of the operation it is a condensate), that was weighed and kept airtight before the analysis. FIG. 2 also gives on the y-axis, the change in the product of the accumulated sublimate mass (m. cum., in g) times 10, over time.

The composition of the consecutive sublimate fractions was analysed by NMR (dissolution in acetone).

The result of these analyses appears in the appended table and in FIG. 3 (where the x-axis gives the accumulated weight of sublimate and the y-axis the weight fraction of each constituent).

It can be seen therein that the 9 g recovered in total contain 8.08 g of lactate groups whereas 10.18 g thereof were introduced at the start. The lactate recovery rate is therefore 79%.

By mixing all the fractions a crude lactide containing 39% by weight of lactide would be obtained.

This figure clearly shows that, at the start of the separation, it is mainly lactic acid that is recovered at the condenser, whilst the lactoyl-lactic acid and the lactide only appear later. It is advantageous to note that the last three samples recovered contain around 90% lactide, which suggests that the subsequent purification described above will be easy.

### Example 3

The purpose of this example is to demonstrate that the starting reactants do not necessarily have to be anhydrous.

In order to do this, the procedure below was followed:

28.1 g of calcium lactate pentahydrate were dissolved in 100 g of water in a 250 ml container, with stirring at 60° C., then 12.8 g of 85% phosphoric acid were added. After 80 minutes of evaporation, a partly crystallized mass remained, which was transferred into a mortar and mixed with 12.5 g of 85% phosphoric acid. It was possible to recover 49.4 g of paste, the consistency of which permitted extrusion using a syringe, the diameter of the tip of which was 5.2 mm.

46.9 g of the rod thus produced were raised in an oven to 50° C. for 18 hours, which reduced the weight to 36.7 g. After coarse milling, 33.7 g of the granules obtained were loaded into the evaporator of the BÜCHI B-585 Kugelrohr minisublimer.

| | | Accumulated | | | | | | | | | |
|---|---|---|---|---|---|---|---|---|---|---|---|
| | Weight | weight | L1A | LD | L2A | L1A | LD | L2A | Lactate | | |
| Fraction No. | g | G | x | x | x | g | g | g | ex-L1A | ex-LD | ex-L2A |
| 1 | 0.7 | 0.7 | 0.9 | 0.03 | 0.07 | 0.63 | 0.021 | 0.049 | 0.504004 | 0.021 | 0.043556 |
| 2 | 2.2 | 2.9 | 0.76 | 0.11 | 0.13 | 1.672 | 0.242 | 0.286 | 1.33761 | 0.242 | 0.254223 |
| 3 | 1.9 | 4.8 | 0.58 | 0.25 | 0.16 | 1.102 | 0.475 | 0.304 | 0.881606 | 0.475 | 0.270223 |
| 4 | 1.7 | 6.5 | 0.48 | 0.4 | 0.13 | 0.816 | 0.68 | 0.221 | 0.652805 | 0.68 | 0.196445 |
| 5 | 0.8 | 7.3 | 0.24 | 0.67 | 0.09 | 0.192 | 0.536 | 0.072 | 0.153601 | 0.536 | 0.064 |
| 6 | 0.5 | 7.8 | 0.11 | 0.83 | 0.05 | 0.055 | 0.415 | 0.025 | 0.044 | 0.415 | 0.022222 |
| 7 | 0.4 | 8.2 | 0.05 | 0.89 | 0.06 | 0.02 | 0.356 | 0.024 | 0.016 | 0.356 | 0.021333 |
| 8 | 0.3 | 8.5 | 0.03 | 0.88 | 0.09 | 0.009 | 0.264 | 0.027 | 0.0072 | 0.264 | 0.024 |
| 9 | 0.6 | 9.1 | 0.02 | 0.92 | 0.06 | 0.012 | 0.552 | 0.036 | 0.0096 | 0.552 | 0.032 |
| Sum | | | | | | 4.508 | 3.541 | 1.044 | 3.606426 | 3.541 | 0.928004 |

x = weight fraction
L1A = lactic acid
LD = cyclic dilactide
L2A = lactoyl-lactate

The device was put under vacuum (5 to 10 mbar) and the setpoint temperature was gradually increased as indicated in FIG. 4 (which in fact represents, on the y-axis, this temperature −100° C.).

At regular intervals, the rotation of the device and the heating were stopped in order to be able to recover sublimate (strictly speaking, at the start of the operation it is a condensate), that was weighed and kept airtight before the analysis. FIG. 4 also gives on the y-axis, the change in the product of the accumulated sublimate mass (m. cum., in g) times 10, over time.

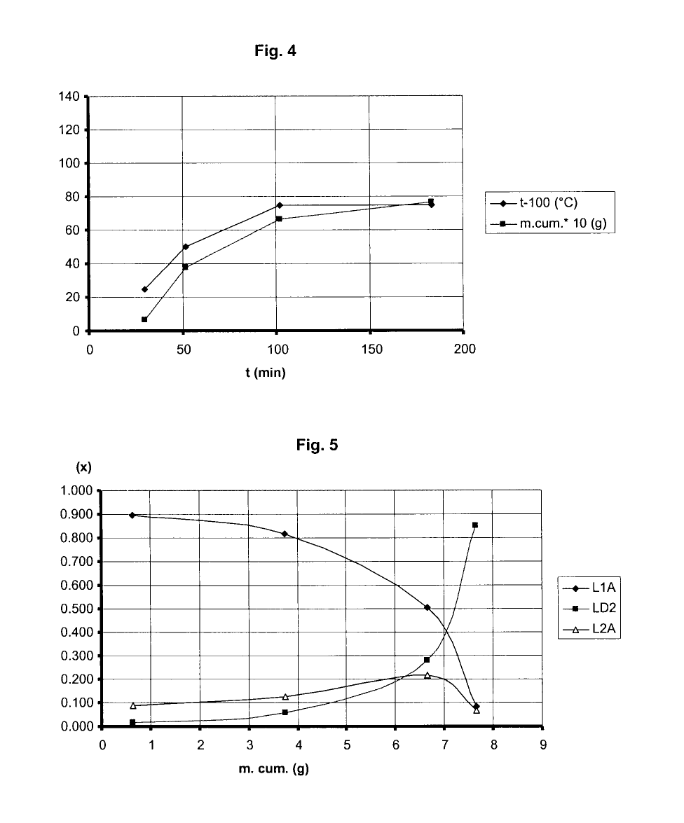

The NMR analysis made it possible to measure the concentrations of the various constituents, either by considering the protons included in the CH₃ group, or by considering the protons bonded to a carbon bearing an alcohol group or an ester group. The averages of these concentrations (x or mass fraction) are given in FIG. 5 for each sample.

It can be seen therein that, at the start of the distillation, mostly lactic acid (L1A) is released, then lactoyl lactic acid (L2A) and lactide (or dilactide, LD2), and that it is only at the end of the separation that the lactide becomes dominant in the sublimate. Therefore, if all the sublimate fractions were mixed together, a mixture would be obtained for which the weight contents would be:

### L1A: 0.631

### LD2: 0.225

### L2A: 0.144

### Example 4

The purpose of this example is to demonstrate that it is possible to add a reaction by-product from a previous batch (here, lactic acid) to the mixture.

In order to do this, the procedure below was followed:

A 250 ml vessel was charged with 55 g of calcium lactate pentahydrate, 42.1 g of 88% lactic acid and 25 g of water. After boiling, whilst stirring, for one hour at atmospheric pressure, the slurry obtained was transferred into a mortar and 50.2 g of 85% phosphoric acid were added thereto. After mixing, the pasty mixture was extruded as a spaghetti that was left to age at ambient temperature for a few days, until becoming a solid that was coarsely milled into granules having an average diameter of 2 to 5 mm.

To prevent these granules from agglomerating during the separation in the mini-sublimer, added to the charge was a small amount of desiccant (5 g of anhydrous monocalcium phosphate powder, obtained by milling the residue of a previous batch) for a charge of 25 g of granules.

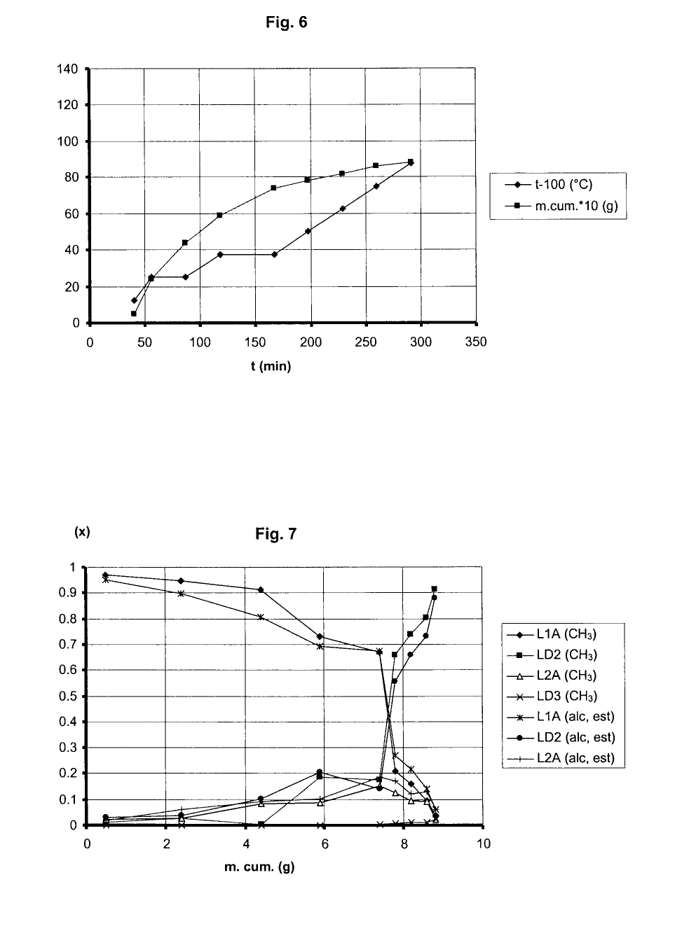

FIG. 6 shows that, under a pressure of 5 to 10 mbar, the separation can be carried out at a relatively low temperature. But in this case (FIG. 7), the lactide concentration of the sublimate only becomes high at the end of the separation.

In FIG. 7, the concentrations obtained by analysis of the NMR signals by the two routes described in the previous example are given.

The weight contents of the mixture of all the sublimate fractions would be:

### L1A: 0.747

### LD2: 0.170

### L2A: 0.082

### LD3: 0.001

### Example 5

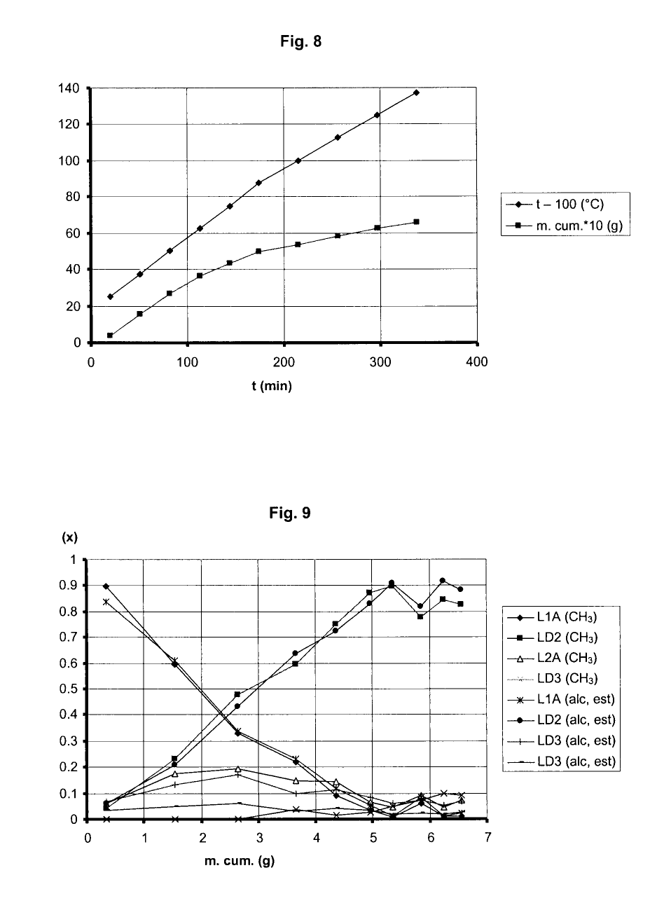

Example 4 was repeated at a higher temperature (compare FIGS. 8 and 6).

In this case the lactide content of the sublimate was increased much more rapidly than in the previous example (compare FIGS. 9 and 7), and the appearance of new NMR peaks was observed, which were attributed to the trilactide (LD3, the cyclic triester).
The average weight contents of the sublimate here would be:

### L1A: 0.300

### LD2: 0.552

### L2A: 0.115

### LD3: 0.033

It therefore appears that, surprisingly, the fact of sublimer at high temperature (typically above 180° C.) makes it possible to favour a high average diester content being obtained.

This application claims priority to European patent application 08158153.0, filed Jun. 12, 2008, incorporated herein by reference.

1—A process for manufacturing a cyclic diester of an α-hydroxy acid comprising:
a salt of a metal and of an α-hydroxy acid is mixed with a strong acid, the pKa of which is less than that of the α-hydroxy acid and the salt of which with the metal is hygroscopic; and
the mixture is left to react at a sufficient temperature and for a sufficient time in order to obtain the cyclic diester dispersed in the hygroscopic salt.

2- The process according to claim 1, where the α-hydroxy acid is lactic acid.

3- The process according to claim 1, where the metal is Ca or Mg.

4- The process according to claim 3, where the salt is obtained by at least partial dehydration of MgLa₂ (Mg lactate) dihydrate or CaLa₂ (Ca lactate) pentahydrate.

5- The process according to claim 1, in which the salt is an anhydrous divalent metal salt.

6- The process according to claim 1, in which the strong acid is phosphoric acid.

7- The process according to claim 5, in which 2 moles of phosphoric acid per mole divalent metal salt are mixed.

8- The process according to claim 1, in which the mixture is subjected to the action of additional hygroscopic salt during and/or after its manufacture.

9- The process according to claim 1, in which the α-hydroxy acid is added to the mixture.

10- The process according to claim 1, in which the mixture is extruded.

11- The process according to claim 1, in which the mixture is subjected to a maturing operation until a solid is obtained.

12- The process according to claim 11, in which the solid is milled and then subjected to distillation/sublimation.

13- The process according to claim 12, in which the sublimation takes place in a chamber comprising a bundle of tubes through which a heat transfer fluid flows, and according to which this fluid is first brought to a temperature below the crystallization temperature of the cyclic diester in order to deposit on this bundle an annular crust of diester containing more volatile impurities; next, the temperature of the fluid is increased gradually to give rise to the melting of a mixture rich in impurities that is recovered in the lower part of the chamber; and finally, the temperature of the fluid is increased sufficiently to melt the purified diester that is also recovered in the lower part of the chamber.

14- The process according to claim 12, in which the sublimation takes place in a chamber that comprises two bundles of tubes and which is designed to be able to operate under vacuum, according to which the heat transfer fluid of the first bundle is first brought to a temperature below the crystallization temperature of the cyclic diester in order to deposit on this bundle an annular crust of diester containing more volatile impurities; next, the heat transfer fluid of the second bundle is brought to a sufficient temperature to give rise to the melting of this surface crust and the chamber is put under vacuum in order to give rise to the fractional distillation of the volatile impurities that it contains; finally, the purified diester is recovered in the lower part of the chamber.

15- The process according to claim 13, according to which a heat exchanger, brought to a temperature above that of the desublimation of the diester but below that of the desublimation of the oligomers of the latter, is placed between the solid and the bundles of tubes so as to thereon condense and recover the oligomers, with a view to the recycling thereof.

16- The process according to claim 14, according to which a heat exchanger, brought to a temperature above that of the desublimation of the diester but below that of the desublimation of the oligomers of the latter, is placed between the solid and the bundles of tubes so as to thereon condense and recover the oligomers, with a view to the recycling thereof.
